(19) United States
(12) Patent Application Publication
(10) Pub. No.: US 2004/0241692 A1
(43) Pub. Date: Dec. 2, 2004

(54) Correction method, apparatus and recording medium on oligonucleotide microarray using principal component analysis

(76) Inventors: Yeoin Yoon, Gyeongsangbuk-do (KR); Jin Hyun Park, Gyeongsangbuk-do (KR)

Correspondence Address: BURNS DOANE SWECKER & MATHIS L L P, POST OFFICE BOX 1404, ALEXANDRIA, VA, 22313-1404, US

(21) Appl. No.: 10/486,225

(22) PCT Filed: Jun. 16, 2003
(86) PCT No.: PCT/KR03/01179

(30) Foreign Application Priority Data
Jun. 18, 2002 (KR) 10-2002-0033864
Jun. 13, 2003 (KR) 10-2003-0038205

(51) Int. Cl. C12Q 1/68; C12M 1/34; B05D 3/00

(52) U.S. Cl. 435/6; 435/287.2; 427/2.11

(57) ABSTRACT

The present invention relates to correction method, apparatus and recording medium on an oligonucleotide microarray using Principal Component Analysis (PCA). More patricularly, the present invention relates to correction method, apparatus and recording medium on the oligonucleotide microarray using the correlation of probe set for detecting and correcting the faulty probe expression data in the outliers of the oligonucleatide microarray by applying PCA to each probe set of gene. Since the faulty probe data is corrected close to the normal value, the present invention makes it possible to remove the noise included in the oligonucleotide microarray, improve the accuracy and efficiency of chip experiment and analysis due to obtainment of accurate expression intensity data, and standardize the oligonucleotide chip data.

### FIELD OF THE INVENTION

The present invention relates to a correction method, an apparatus and a recording medium on an oligonucleotide microarray using Principal Component Analysis(PCA). More particularly, the present invention relates to the correction method, the apparatus and the recording medium on the oligonucleotide microarray using the correlation of probe set for detecting and correcting the faulty probe expression data in the outliers of the oligonucleotide microarray by applying PCA to each probe set of gene.

### BACKGROUND OF THE INVENTION

The oligonucleotide microarray means data extracted from the oligonucleotide chip. The oligonucleotide chip made scientist's ultimate goal possible to find genetic mutations related to diseases and new medicine. Also, the oligonucleotide chip, as an outstanding system for research on the genetics, can be applied to disease researches such as cancer, Alzheimer and aging, or other various applications. Affymetrix GeneChipe®, a high-density oligonucleotide chip, is DNA array that hundred thousands of probes composed of 20˜25 mer oligonucleotides are directly synthesized by a base on the array in different forms. The probes are synthesized in a small glass plate having dimensions about 1.28 cm², and photolithography and solid-phase chemistry are employed in manufacturing array of Affymetrix GeneChip®. One gene in the oligonucleotide chip is composed of 16 to 20 probe pairs consisting 20 to 25 mer oligonucleotide, and the probe is designed to maximize the accuracy, the singularity and the reproducibility, so that it can distinguish signal and background of target having similar sequence.

As shown in FIG. 1, each probe set of the oligonucleotide chip consists of about 20 probe pairs, and indicates transcription information of one gene or sequence. Generally, the probe pair consists of perfect match(PM) having target RNA sequence and mismatch(MM) having one sing nucleotide difference at the middle of the sequence. PM corresponds to signal and MM corresponds to background.

The data obtained from the oligonucleotide microarray are huge data, and therefore, computer aided analysis and statistical analysis is required. Most of microarray data analyses are performed at the gene expression level rather than the probe expression level, and as the oligonucleotide chip represents transcription information of one gene by probe expression data of about 20 probe pairs, so that it is complicated to analyze because probe data of about 20 pairs have to be simplified to one value through a specific process.

On the other side, the oligonucleotide chip experiment requires very experienced technique and is exposed to many error or noise through a whole process. Since the measurement error, which occurs during manufacturing the oligonucleotide chip, is more than 15% of total data, it is very important to detect measurement error and correct them. If the measurement error is not examined accurately, it may lead a faulty result in the analysis. The contaminated data due to the measurement error are associated with the efficiency and the accuracy of bio-tech experiment that requires enormous cost, time and human resources. This is a serious problem in that the analysis and decision using the oligonucleotide chip is closely related to the health, clinic and life. Further, even if the measurement error is recognized accurately, it was common that not only the error data but also other data related to the error were discarded for maintaining an objective level of significance and credibility of data analysis because a technique to correct the error is not developed yet.

The aforementioned measurement errors occur in the following cases: 1) sample itself is contaminated, 2) the spot is spoiled, 3) defects in experimental equipment and process, 4) hybridization is not completed correctly, and 5) cross-hybridization occurs. The aforementioned measurement errors affect a specific probe to get an abnormal expression value, and therefore, the probe shows a different pattern from the expression pattern of other normal probes. The sample having an abnormal probe expression value is called “outlier” in the oligonucleotide chip.

There have been some conventional methods for examining or correcting the outlier: 1) to find MM larger than PM and to correct the value of MM much smaller than that of PM, 2) to calculate discriminant score(=(PM−MM)/(PM+MM)) for each probe pair, and to classify probe pairs into “Present(≧0.015)” or “Absent(<0.015), and to convert “Absent” probe into 0. However, the limits of the aforementioned method are as follows: 1) the method can only detect abnormality in some specific cases, 2) in most cases, the method produces biologically-meaningless value because simple statistical method is used. Thus, the aforementioned method is not suitable for the error correction but just a temporary remedy. Also, according to another conventional method, some data that are not satisfied to a given condition, namely, outliers were excluded from the analysis, however, this method 1) may degrade the efficiency of the experiment because the size of data obtained from the experiment is reduced, which results in the information loss. 2) may cause the experiment to be failure if the measurement error affects data representing principal target genes, 3) may result in useless analysis because the given conditions are established statistically without considering biological characteristics. (https://www.affymetrix.com/Download/manuals/data analysis fundamentals manual.pdf; Cheng Li, et. al., Model-based analysis of oligonucleotide arrays: Expression index computation and outliers detection, PNAS, 98:31-36, 2001).

Data analysis of the oligonucleotide microarray can be divided into a low-level analysis and a high-level analysis. The low-level analysis refers to a standardization of the microarray data performed before entering the substantial analysis such as a feature extraction from the experiment, a contamination filtering, a data normalization, etc. Since the high-level analysis can be performed only after the low-level analysis is accomplished well, many researches on the low-level analysis are conducted and the low-level analysis can be treated as a fundamental technology. The high-level analysis is a method for inducing intended result just as intended through clustering, classification, discriminant analysis, etc. on the data normalized at the low-level analysis, and many techniques on high-level analysis are already established and well-known. As the performance of the high-level analysis is dependent on the result of low-level analysis, the developed countries invest human resources and money tremendously in developing the fundamental technologies of the low-level analysis.

### DETAILED DESCRIPTION OF THE INVENTION

Accordingly, the object of the present invention is to provide a correction method on an oligonucleotide microarray using PCA.

Another object of the present invention is to provide a recording medium, including a program containing computer-executable instructions to perform a correction method for correcting an outlier on an oligonucleotide microarray using PCA.

Still another object of the present invention is to provide a correction apparatus on an oligonucleotide microarray using PCA.

Still another object of the present invention is to provide a computer program, being executed by a digital processing device, for performing the correction method on an oligonucleotide microarray using PCA.

To achieve aforementioned objects, the present invention may provide a method for correcting outliers on an oligonucleotide microarray using PCA(principal component analysis), said method comprising the steps of:

(a) constructing a correlation structure model indicating a correlation structure between probes of the oligonucleotide microarray data by use of the PCA; and

(b) correcting a faulty probe data by projecting said correlation structure model to the outlier.

The present invention further may comprise the step of constructing data matrix of each probe set of genes for the oligonucleotide microarray data, wherein said correlation structure model may include the correlation structure of the probes included in said data matrix.

Also, the present invention may provide a method for correcting outliers on an oligonucleotide microarray using PCA (principal component analysis), said method comprising the steps of:

(a) detecting an outlier in the oligonucleotide microarray data;

(b) constructing a first correlation structure model for model data to be used to correct the outlier by use of PCA;

(c) correcting a faulty probe data by projecting said first correlation structure model to the outlier.

The step (a) may comprise the step of constructing a second correlation structure model of probes of each probe set by use of the PCA; and detecting the outlier through calculating SPE index between the model value from said second correlation structure model and the raw data value.

Also, the present invention may provide a computer-readable medium including a program containing computer-executable instructions to perform a correction method for correcting outliers on an oligonucleotide microarray using PCA(principal component analysis), wherein the program performs the steps of:

(a) constructing a correlation structure model indicating a correlation structure between probes of the oligonucleotide microarray data by use of the PCA; and

(b) correcting a faulty probe data by projecting said correlation structure model to the outlier.

The present invention further may perform the step of constructing data matrix of each probe set of genes for the oligonucleotide microarray data, wherein said correlation structure model includes the correlation structure of the probes included in said data matrix.

Also, the present invention may provide a computer-readable medium including a program containing computer-executable instructions to perform a correction method for correcting outliers on an oligonucleotide microarray using PCA(principal component analysis), wherein the program performs the steps of:

(a) detecting an outlier in the oligonucleotide microarray data;

(b) constructing a first correlation structure model for model data to be used to correct the outlier by use of PCA;

(c) correcting a faulty probe data by projecting said first correlation structure model to the outlier.

The step (a) may comprise the steps of constructing a second correlation structure model of probes of each probe set by use of the PCA; and detecting the outlier through calculating SPE index between the model value from said second correlation structure model and the raw data value.

The present invention may provide a correction apparatus of an oligonucleotide microarray, said apparatus comprising:

a correlation structure model generator for constructing a correlation structure model indicating a correlation structure between probes of the oligonucleotide microarray data by use of the PCA; and

a data corrector for correcting a faulty probe data by projecting said correlation structure model to the outlier.

The correlation structure model generator may generate a data matrix of each probe set of each gene for the oligonucleotide microarray data and constructs the correlation structure model of the probes included in the data matrix.

The present invention may provide a correction apparatus of an oligonucleotide microarray, said apparatus comprising:

an outlier extractor for detecting outlier from the oligonucleotide microarray data

a first correlation structure model generator for constructing a first correlation structure model for model data to be used to correct the outlier by use of the PCA; and

a data corrector for correcting a faulty probe data by projecting said first correlation structure model to the outlier.

The outlier extractor may comprise means for constructing a second correlation structure model of probes of each probe set by use of the PCA; and means for detecting the outlier through calculating SPE index between the model value from said second correlation structure model and the raw data value.

The present invention may provide a program being executed in a digital processing device to perform a correction method on an oligonucleotide microarray, wherein said program controls operations of the digital processing device to perform the correction method, said program performs the steps of:

(a) constructing a correlation structure model indicating a correlation structure between probes of the oligonucleotide microarray data by use of the PCA; and

(b) correcting a faulty probe data by projecting said correlation structure model to the outlier.

The present invention may provide a program being executed in a digital processing device to perform a correction method on an oligonucleotide microarray, wherein said program controls operations of the digital processing device to perform the correction method, said program performs the steps of:

(a) detecting an outlier in the oligonucleotide microarray data;

(b) constructing a first correlation structure model for model data to be used to correct the outlier by use of PCA;

(c) correcting a faulty probe data by projecting said first correlation structure model to the outlier.

In the present invention, the term “probe set of each gene” is meant to include data of perfect match probe(PM) and mismatch probe(MM), both indicating transcription information of a specific gene, or data of the selected principal probes.

### BRIEF DESCRIPTION OF THE DRAWINGS

FIG. 1 shows the structure of the probe set of each gene on the oligonucleotide chip.

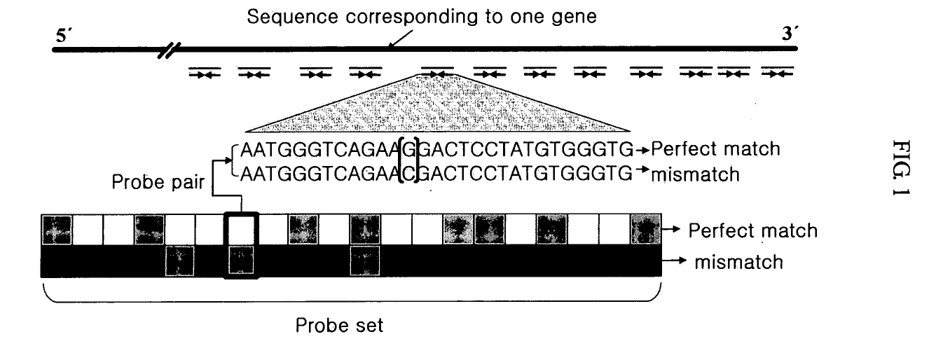

FIG. 2 is a flowchart showing the correction method on the oligonucleotide microarray using PCA when the outliers are unknown.

FIG. 3 shows the three-dimensional data structure of microarray of the oligonucleotide chip(FIG. 3a) and the two-dimensional data structure of probe set of a specific gene originated from the three-dimensional data structure(FIG. 3b).

FIG. 4 shows the method for arranging data according to each probe set.

FIG. 5a shows the raw expression value of 40 probes(18 samples×40 probes) of ‘ribosomal protein L8 gene’ probe set.

FIG. 5b shows the score chart of applying PCA to ‘ribosomal protein L8 gene’ probe set when the number of PC is 2.

FIG. 5c shows the loading chart of applying PCA to ‘ribosomal protein L8 gene’ probe set.

FIG. 6a shows SPE chart according to the correlation structure model of ribosomal protein L8 gene probe set.

FIG. 6b shows SPE chart that is corrected by use of the model constructed with all samples of ‘ribosomal protein L8 gene’.

FIG. 6c shows SPE chart that is corrected by use of the model constructed with samples of specific group where outlier should be included after correction or samples having value close to outlier.

FIG. 7 shows the result of correction on the faulty probe expression value of ‘ribosomal protein L8 gene’ according to the present invention.

FIG. 8a shows the results of performing a single linkage method, one of hierarchical clustering, on the raw data of human fibroblast cell including the outlier according to the present invention.

FIG. 8b shows the results of performing an average linkage method, one of hierarchical clustering, on the raw data of human fibroblast cell including the outlier according to the present invention.

FIG. 9a shows the results of performing a single linkage method, one of hierarchical clustering, after correction on the faulty probe expression value of the raw data of human fibroblast cell according to the present invention.

FIG. 9b shows the results of performing an average linkage method, one of hierarchical clustering, after correction on the faulty probe expression value of the raw data of human fibroblast cell according to the present invention.

### BEST MODE FOR CARRYING OUT THE INVENTION

Hereinafter, the present invention will be described with embodiments in detail. But, the following description is just examples of the present invention, so the scope and sprits of the present invention are not limited to the description.

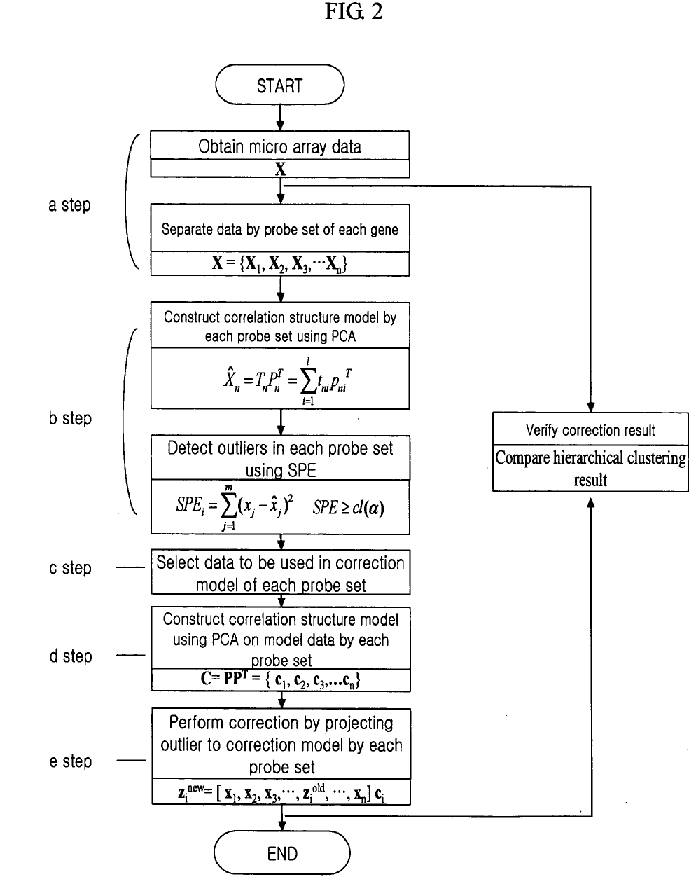

In an embodiment of the present invention, the correction method on the oligonucleotide microarray using PCA is employed in order to correct the outliers when the outliers are unknown. The whole procedure of the correction method on the oligonucleotide microarray using PCA is shown in FIG. 2.

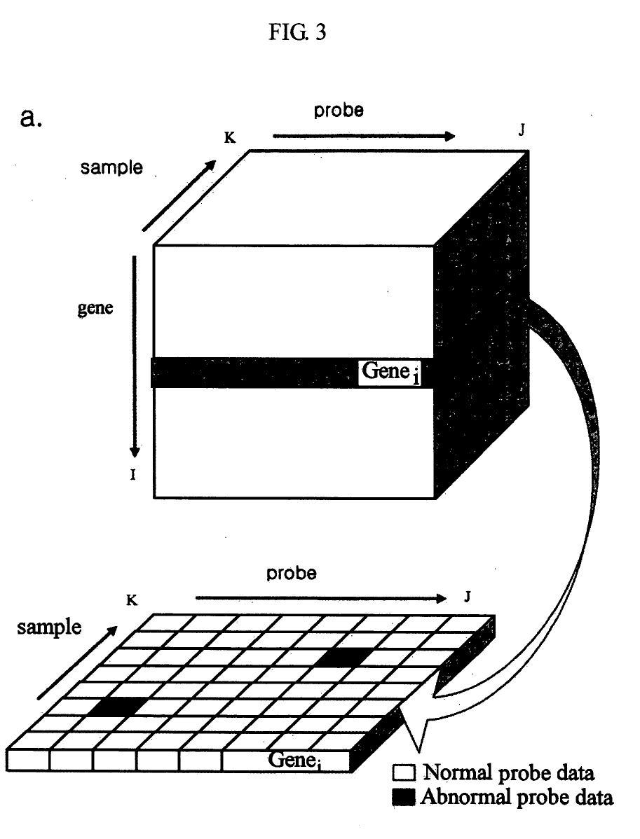

As shown in FIG. 2, the correction method on the oligonucleotide microarray using PCA comprises the step of obtaining microarray data and reconstructing data matrix by the probe set of each gene. Namely, as shown in FIG. 3, this step transforms the three-dimensional oligonucleotide microarray data into two-dimensional matrix. The three-dimensional oligonucleotide microarray data X is a matrix composed of ‘n genes×i probes×k samples’ and can be reconstructed by two-dimensional data matrix Xₙ for each probe set of gene, for example ‘i probes×k samples’. The three-dimensional oligonucleotide microarray data X can be represented as Equation 1.

X=[X₁, X₂, X₃, . . . , Xₙ] Equation 1

And as shown in step b of FIG. 2, the present invention comprises a step of constructing correlation structure model of probes included in the probe set by applying PCA to data matrix of each probe set and of detecting outliers. As the probe expression values of 20 pairs have an intense correlation with each other because these values represent data about one gene, it is preferable to construct the correlation structure model to be corrected by applying PCA.

The PCA, as an analysis on complex data of multivariate, reduces the variables by use of correlation between variables to extract principal data. (Hwang. D. H, et al. Online monitoring technique using multivariate statistical method, 15(3), 1997).

The PCA can be expressed by the mathematical model of algorithm as follows:

X=TP^{T}+E Equation 2

Where, P represents a loading matrix, T represents a score matrix, E represents error, and superscript T represents a transpose.

After defining the correlation structure model by applying PCA, \hat{X}ₙ that can be predicted from the correlation structure model is obtained. In the present invention, the correlation structure model may be a covariance matrix comprising correlation data of probes included in each probe set. The covariance matrix C can be obtained by Equation 3.

$$C = \frac{1}{k - 1} ( {X}_{n}^{T} {X}_{n} ) \qquad Equation 3$$

(Xₙ is n^{th} probe set of two dimension, k is the number of samples)

The elements in the covariance matrix refer to variance coefficient or correlation coefficient. As a basic index for measuring the linear correlation between two variables a and b, the elements in the covariance matrix indicate the degree of change of two variables a and b toward same direction based on each mean.(Chun. C. H, et al, Applied Engineering Statistics, POSTECH PRESS, 1998)

After calculating an eigenvector of covariance matrix, Pₙ, the score vector Tₙ can be calculated by projecting Xₙ to Pₙ. \hat{X}ₙ can be calculated by a product of score vector Tₙ and cigenvector Pₙ. This can be represented as follows.

$${\hat{X}}_{n} = {T}_{n} {P}_{n}^{T} = {\sum }_{i = 1}^{l} {t}_{{n}_{i}} {p}_{{n}_{i}}^{T} \qquad Equation 4$$

The eigenvector is a set of coefficients of probes having correlation to each other, and it can be obtained from SVD(Singular Value Decomposition), and NIPALS(Nonlinear Iterative Partial Least Square), etc.

After defining the correlation structure model and obtaining \hat{X}ₙ that is predicted from the correlation structure model, the outlier is detected by comparing the difference between the raw data values of each sample, Xₙ, and the predicted data value \hat{X}ₙ. SPE(Squared Prediction Error), which is used at this step, represents the squared perpendicular distance of probe data that are projected on the model space, it means the distance between the model data and the raw data. Thus, the sample having large SPE value can be defined as an outlier. (Jackson, J. E., A user's guide to principal components, 1991, Wiley, Newyork)

The SPE value of k^{th} sample is calculated by the following Equation 5.

$${SPE}_{k} = {\sum }_{j = 1}^{m} {( {x}_{kj} - {\hat{x}}_{kj} )}^{2} \qquad Equation 5$$

Where, xₖⱼ represents the raw value of j^{th} probe, and \hat{x}ₖⱼ represents the model value of j^{th} probe.

SPE≧cl(α)

Where, cl represents the reference value of the confidence limit in the model and α represents distribution, and the outlier can be detected by finding the sample deviated from the confidence limit.

After detecting outlier, as shown in step c of FIG. 2, the present invention fuirther comprises the step of selecting data for constructing correlation structure model that is used to correct the outlier of each probe set of each gene. Because the correlation between the normal sample data is no- fully reflected in the correlation structure model if the outlier is included, the normal sample data must be selected to construct the ideal correction model. At this step, the model data are selected from the sample in the group close to the value of outlier. Namely, according to the pattern of the normal sample in the correlation structure model space for each probe set of each gene, if the sample data are located in one place, the correlation structure model may be constructed with all of the collected normal data, and if these sample data are divided into two or three groups, the correlation structure model may be constructed with samples of the group where outlier should be included after correction.

Subsequently, the present invention comprises the step of constructing the correlation structure model by applying PCA to the selected model data as shown in step d of FIG. 2.

The correlation structure model may be the covariance matrix including the correlation data of the probes included in each probe set, and is constructed by applying PCA to the selected model data.

Let T=XP in the model space,

T≈TP^{T}=XPP^{T} Equation 7

Where, assuming PP^{T} to be the covariance matrix C, C is expressed as follows:

C≈PP^{T}=[c₁, c₂, . . . , cᵢ] Equation 8

Where, the i^{th} probe can be expressed as follows:

Cᵢ^{T}[c₁ᵢ, c₂ᵢ, . . . , cᵢᵢ] Equation 9

The covariance matrix C summarizes the correlation between each variable, so faulty probe intensity can be corrected by projecting outlier on this covariance matrix C.

Finally, the present invention comprises the step of correcting faulty data by projecting the correlation structure model to the detected outliers as shown in step e of FIG. 2. Projection of the correlation structure model to the outlier refers to a product of the data matrix comprising outlier and the covariance matrix comprising the correlation data. The faulty probe data is corrected by iterating the projection until the value of outlier data converges.

The raw data matrix Xₙ can be expressed by column vector as follows:

Xₙ=[x₁, x₂, . . . , zᵢ, . . . xᵢ] Equation 10

Where, zᵢ is a row vector including the probe data to be corrected.

The value of zᵢ is corrected by projecting the covariance matrix cᵢ to Xₙ

The Equation of the corrected i^{th} probe \hat{z}ᵢ is expressed as follows:

\hat{z}ᵢ=[x₁, x₂, . . . , zᵢ, . . . x₁]x₁c₁

If specific probe zᵢ is projected to the covariance matrix cᵢ, the faulty probe data are changed to the values inclined to the correlation structure. If the changed values are projected on cᵢ for correction again, the values more inclined to the correlation structure are generated, and through repetition of projection until the value of zᵢ is not changed any more(\hat{z}ᵢ−z₁≈0), finally the faulty probe data is corrected to the value corresponding to the correlation structure.

X_{c}, the probe set data matrix having the corrected i^{th} probe, can be expressed as follows:

X_{c}=[x₁, x₂, . . . , \hat{z}₁, . . . xᵢ]

In another embodiment of the present invention, if the outliers are known, step b and step c of FIG. 2 can be omitted. Namely, the correlation structure model of probes included in the probe set of each gene can be constructed through obtaining the oligonucleotide microarray data, constructing data matrix of each probe set of each gene and applying PCA to the data matrix of each probe set. And, the faulty probe data can be corrected by projecting the correlation structure model on the outlier.

In still another embodiment of the present invention, the public oligonucleotide chip data are applied to the method in FIG. 2. The public data used in aforementioned embodiment are presented from the statistical gene lab of human gene department in Ohio State University by using Affymetrix oligonucleotide chip. (Lemon, W J, et. al., Theoretical and experimental comparison of gene expression estimators for oligonucleotide arrays, Bioinformatics 2001).

The aforementioned data are 18 sample data obtained from the 6 times experiments with the Affymetrix oligonucleotide chips on three human fibroblast cell groups, which are a FBS(fatal bovine serum) starved group A, a serum stimulated group C, and a 50:50 mixture of starved/stimulated group B. As FBS is absent in group A(sample 1 to 6), cell division almost stops in Go phase among cell cycles and therefore cell proliferation does not occur and metabolic activity becomes lower. For FBS is present in group C(sample 13 to 18), there are cell divisions-due to the vigorous cell cycle and most of cells have high-metabolic activity. Group B(sample 7 to 12) has a intermediate characteristic of group A and group C.

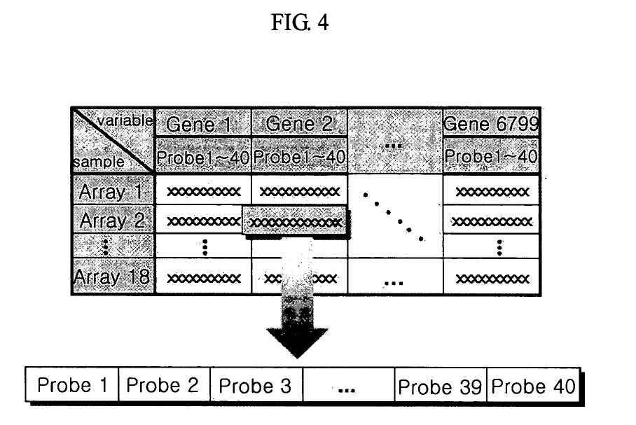

The human fibroblast cell data comprises data of 18 samples and 6799 gene probe sets, and each gene probe set has 40 probe expression values. In order to perform the correction method on the oligonucleotide microarray using correlation on probe sets in the present invention, the data are arranged according to each probe set as shown in FIG. 4.

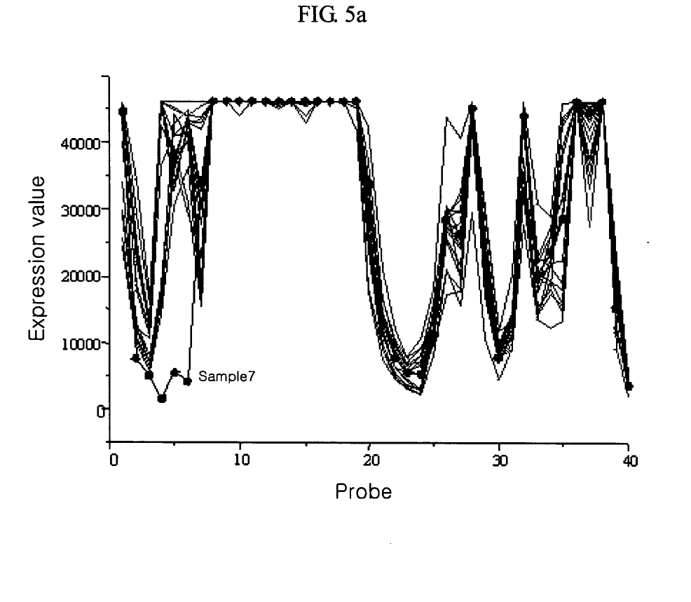

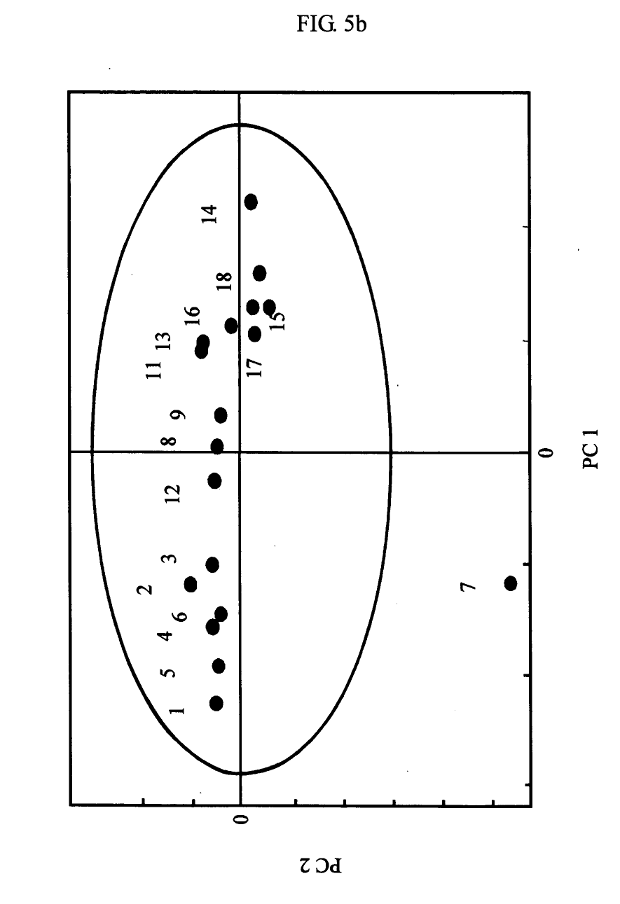

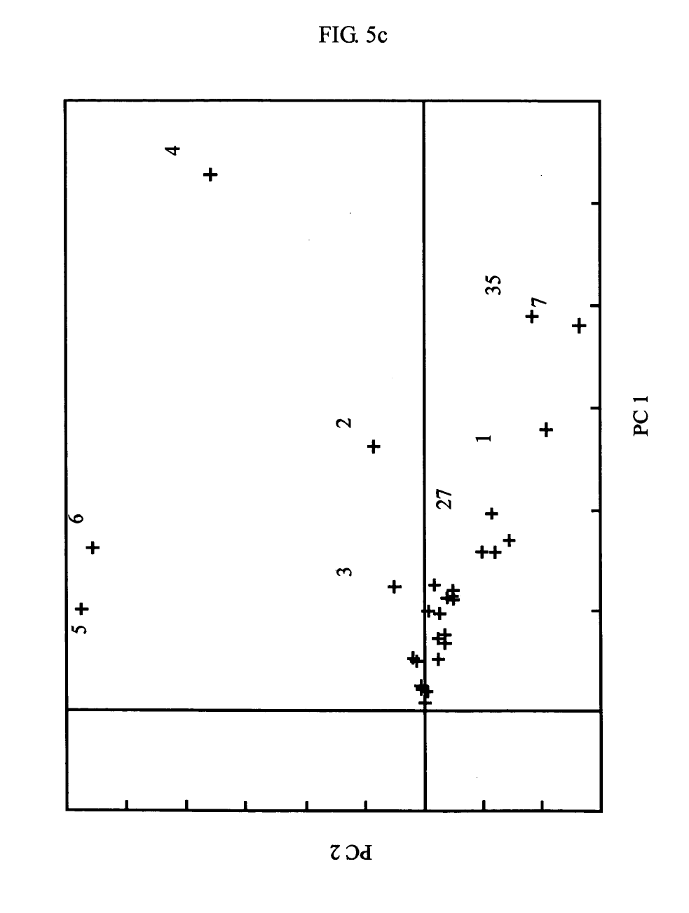

Hereinafter, the present invention will be described with an example of ‘ribosomal protein L8 gene’ of probe set X_{L8}, which is selected as one set among 6799 gene probe sets. The selected gene set is composed ‘18 samples by 40 probes’ as shown in FIG 5a.

To detect outlier, let apply PCA to each probe set to define correlation structure of each probe set, and the sample having different pattern to correlation structure from other sample is regarded as outlier.

If the correlation structure of raw data is defined by PCA, as shown in FIG. 5b and FIG. 5c, the score value and the loading value are generated. The score value indicates the value of each samples on the new PC axis(new variable generated by the correlation), and the loading value indicates the raw variables contribution degree to PC.

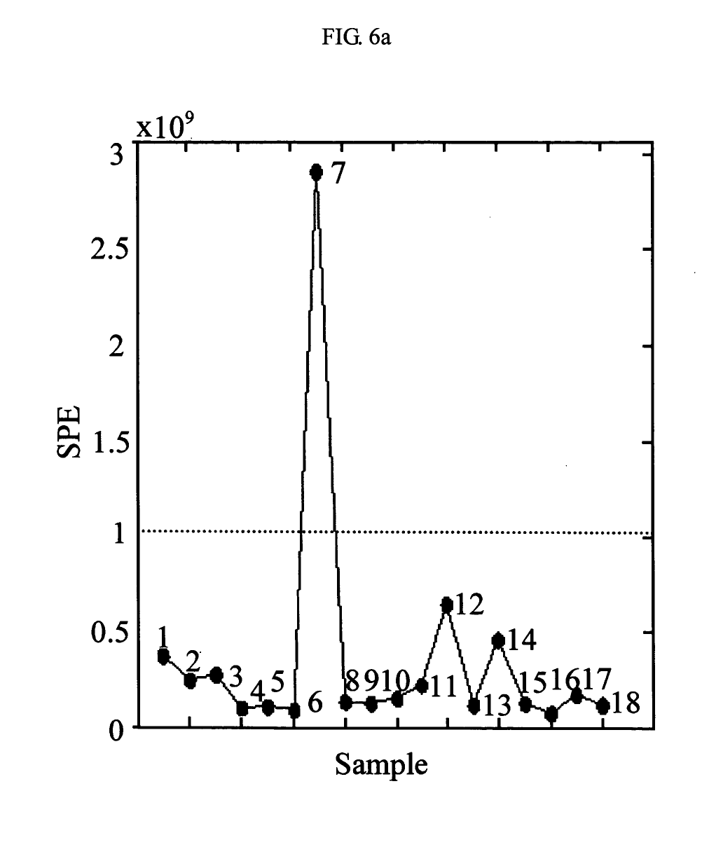

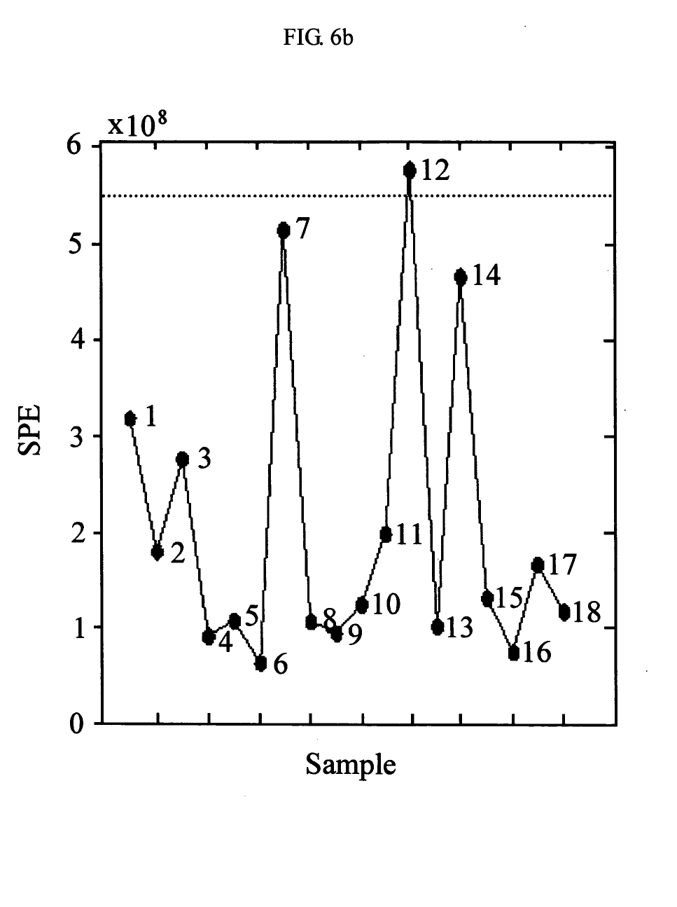

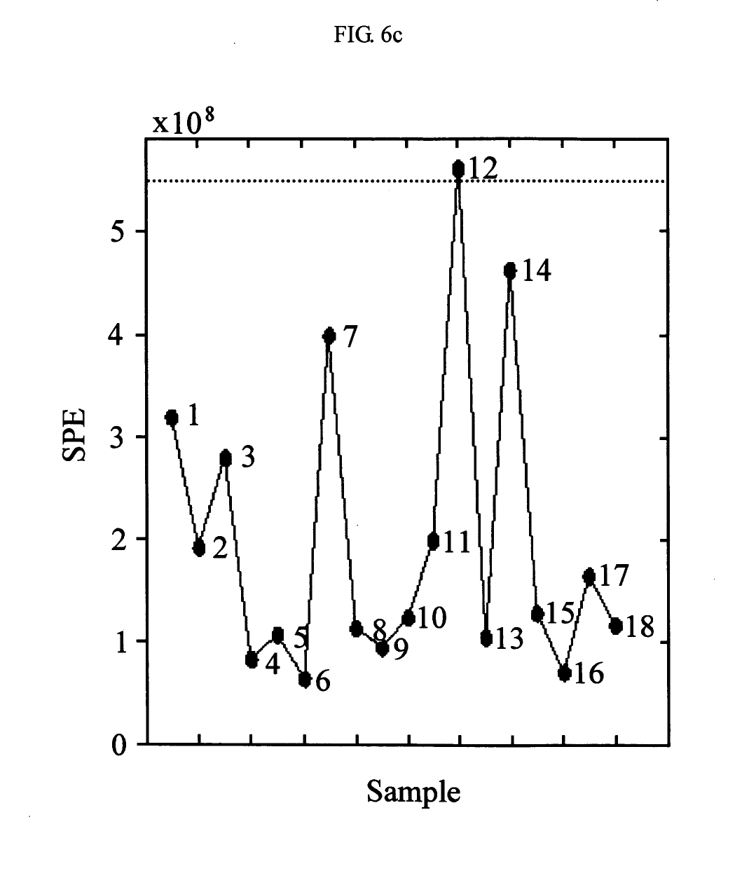

The score chart of first two principal components is shown in FIG. 5b. The samples locating close to each other have similar pattern, and the samples locating far from each other indicate different patterns. The score chart shows the score values of the first principal component and the second principal component. Also, statistical confidence limit with a significance level, 0.05(circular solid line in FIG. 6b) is calculated in the score chart, and outlier is identified based on the confidence limit.

As the score value of sample 7 exceeds the confidence limit, it is regarded as outlier. In the loading chart of FIG. 5c, the contribution of the probe for the first principal component(the largest principal component) and the second principal component(the second largest principal component) of the correlation structure model can be measured by the loading coefficients.

After defining the correlation structure, outlier is detected by use of SPE index. In FIG. 6a showing SPE index of the sample for ribosomal protein L8 gene before correction, the sample 7 located outside of the statistical limit has different pattern from other samples.

To construct the correlation structure model to be used for correcting the detected outlier, the normal sample data are required. The pattern of the normal samples in the correlation structure model space of each gene probe set may change. Even in the embodiment, all 18 samples concentrate at the same point in the model space of a certain probe set, and samples are divided into two or three groups in another probe set. The selection of modeling samples can be changed according to distribution of these samples. Thus, if all samples concentrate at the same point, the model may be constructed with all samples(method 1). If the samples are divided into two or three groups, the model may be constructed with samples of specific group where outlier should be included after correction or with samples having value close to outlier(method 2).

In the embodiment of the present invention, models corresponding to the two methods were constructed.

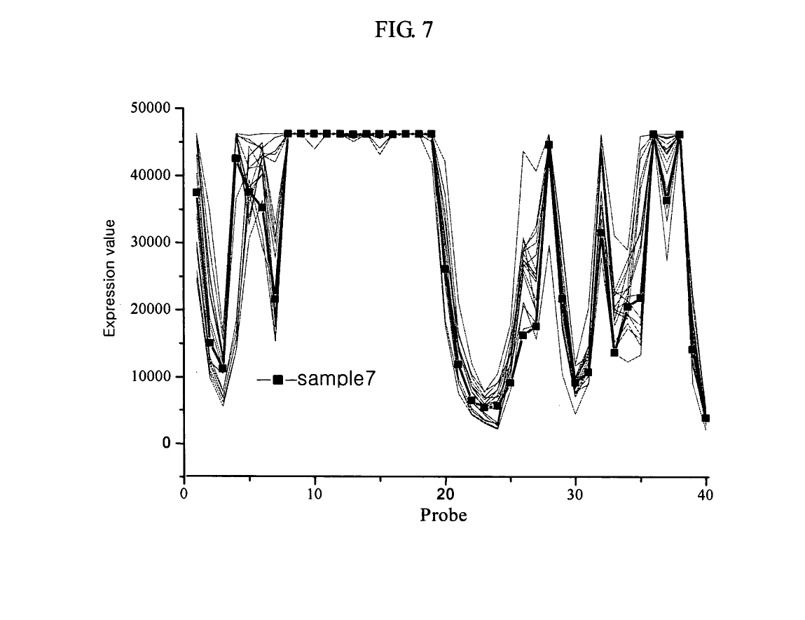

By projecting the sample 7, which is determined to be outlier, on the correction model, probe data 4, 5, 6 are corrected to the values which are close to the normal values. FIG. 6b shows SPE chart that is corrected by use of the model constructed with all normal sample data according to the method 1. FIG. 6c shows SPE chart that is corrected by use of the model constructed with samples of specific group where outlier must be included after correction or samples having value close to outlier according to the method 2. In FIG. 6b and FIG. 6c, which show the result of correction according to the present invention through SPE index, the sample 7 appears below the reference line. It means that the sample 7 becomes similar to other samples in the appearance. Namely, it means that the correction is completed successfully. This can be proved in FIG. 7, which shows the probe set of ‘ribosomal protein L8 gene’ after correction. Comparing FIG. 7 with FIG. 5a, the sample 7, which was located far from other samples before correction, is located close to the normal samples after correction.

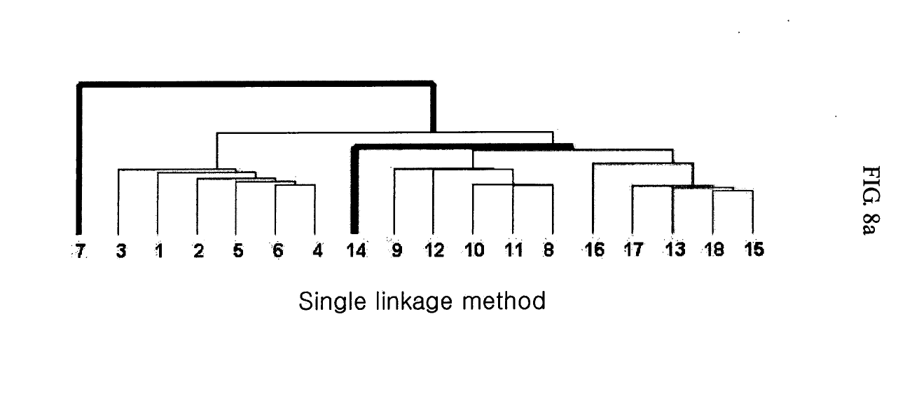

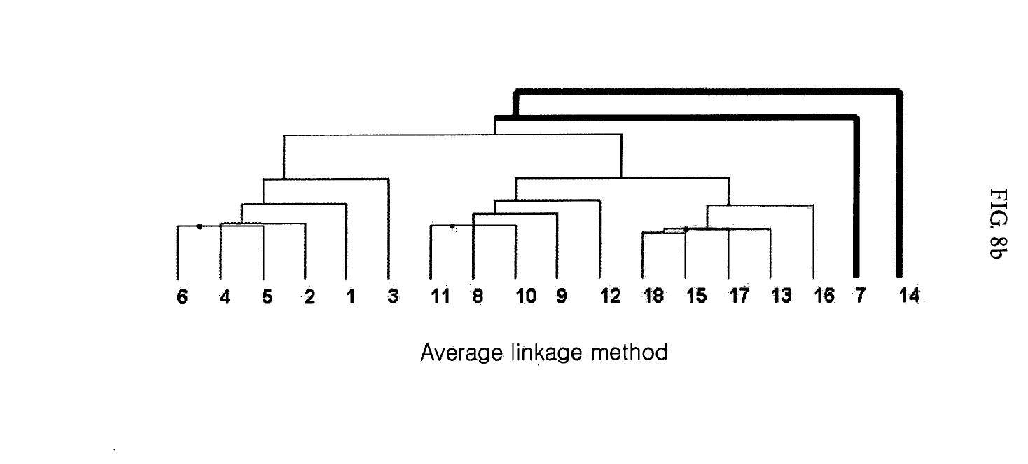

To verify the performance of the correction method that is proposed by the present invention more objectively, the preferred embodiment of the present invention performs a hierarchical clustering method, one of high-level analysis, with the pre-correction data and the post-correction data, and compares the results of the clustering. When the data are clustered, 18 samples must belong to the groups where the samples were identified in the experiment process. FIG. 8a and FIG. 8b show the results of performing hierarchical clustering on the pre-correction data, and more particularly, FIG. 8a is the result of applying a single linkage method and FIG. 8b is the result of average linkage method. As shown in FIG. 8a and FIG. 8b, the sample 7 and 14 are detected as outliers. Since the sample 7, which should be included in group b, is included in the other group (A or C) or is not included to any groups, it indicates that the sample 7 is abnormal.

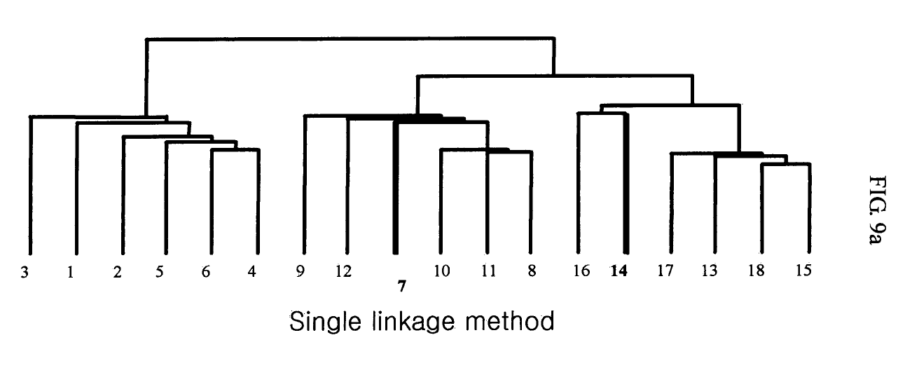

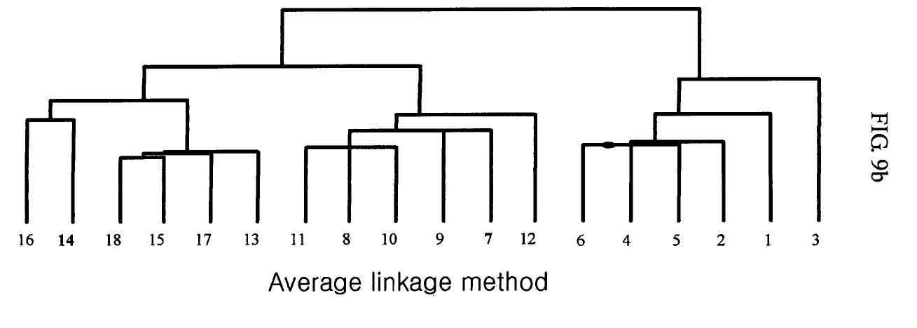

Meanwhile, FIG. 9a and FIG. 9b show the results of performing hierarchical clustering on the whole gene probe sets. FIG. 9a is the result of applying a single linkage method and FIG. 9b is the result of applying average linkage method

When comparing FIG. 9a and 9b with FIG. 8a and 8b, it is corroborated that after performing correction method of the present invention, sample 7 and 14 are included in the group B and C where these samples should be included. Thus, the performance of the correction method on probe data using the correlation according to the present invention is verified.

The present invention is not limited to the process methods specified by the aforementioned embodiments, and it is apparent that those who can apply all kind of mathematical/statistical methods using correlation between probe data can modify or improve the present invention without departing from the sprits and the scope of the present invention.

### Industrial Applicability

As described above, through detecting outlier with the correlation between 40 probe pairs that consist of one probe set and correcting the outlier with the correlation structure model, the correction method, apparatus and recording medium on the oligonucleotide microarray using PCA of the present invention can preserve the biological characteristics of the raw data and improve the significancy and credibility of data.

Also, since the present invention is applied to the probe set of each gene, it is possible to correct data having difference library with same correction model if these data have common probe set.

Also, through performing the present invention, it is possible not only to improve the performance of the high-level analysis but also to reduce the analysis error. Also, through performing the present invention, it is possible to effectively standardize the data experimented at several places due to the aforementioned effects.

1. A method for correcting outliers on an oligonucleotide microarray using PCA (principal component analysis), said method comprising the steps of:
(a) constructing a correlation structure model indicating a correlation structure between probes of the oligonucleotide microarray data by use of the PCA; and
(b) correcting a faulty probe data by projecting said correlation structure model to the outlier.

2. The method of claim 1, further comprising the step of constructing data matrix of each probe set of genes for the oligonucleotide microarray data, wherein said correlation structure model includes the correlation structure of the probes included in said data matrix.

3. The method of claim 2, wherein said correlation structure model is a covariance matrix comprising the correlation data of the probes.

4. The method of claim 3, wherein said covariance matrix is calculated by use of a loading matrix and a transpose matrix of the loading matrix in PCA.

5. The method of claim 1, wherein the projection of said correlation structure model to the outlier in said step (b) is a product of the data matrix including the outlier and said covariance matrix including the correlation data, wherein said step (b) is iterated until the outlier data value converges in order to correct the faulty probe data.

6. A method for correcting outliers on an oligonucleotide microarray using PCA (principal component analysis), said method comprising the steps of:
(a) detecting an outlier in the oligonucleotide microarray data;
(b) constructing a first correlation structure model for model data to be used to correct the outlier by use of PCA;
(c) correcting a faulty probe data by projecting said first correlation structure model to the outlier.

7. The method of claim 6, wherein said step (a) comprises the steps of:
constructing a second correlation structure model of probes of each probe set by use of the PCA; and
detecting the outlier through calculating SPE index between the model value from said second correlation structure model and the raw data value.

8. The method of claim 6, wherein said first correlation structure model is the correlation structure of the probes included in each probe set of genes.

9. The method of claim 6, wherein said first correlation structure model and said second correlation structure model are covariance matrix comprising the correlation data of the probes included in each probe set.

10. The method of claim 9, wherein said covariance matrix is calculated by use of a loading matrix and a transpose matrix of the loading matrix in PCA.

11. The method of claim 6, wherein the model data in said step (b) is selected from the samples of a group close to the value of the outlier.

12. The method of claim 6, wherein the projection of said correlation structure model to the outlier in said step (c) is a product of the data matrix including the outlier and said covariance matrix including the correlation data, wherein said step (c) is iterated until the outlier data value converges in order to correct the faulty probe data.

13. A computer-readable medium including a program containing computer-executable instructions to perform a correction method for correcting an outlier on an oligonucleotide microarray using PCA(principal component analysis), wherein the program performs the steps of:
(a) constructing a correlation structure model indicating a correlation structure between probes of the oligonucleotide microarray data by use of the PCA; and
(b) correcting a faulty probe data by projecting said correlation structure model to the outlier.

14. The computer-readable medium of claim 13, further performing the step of constructing data matrix of each probe set of genes for the oligonucleotide microarray data, wherein said correlation structure model includes the correlation structure of the probes included in said data matrix.

15. The computer-readable medium of claim 13, wherein said correlation structure model is a covariance matrix comprising the correlation data of the probes.

16. The computer-readable medium of claim 13, wherein the projection of said correlation structure model to the outlier in said step (b) is a product of the data matrix including the outlier and said covariance matrix including the correlation data, wherein said step (b) is iterated until the outlier data value converges in order to correct the faulty probe data.

17. A computer-readable medium including a program containing computer-executable instructions to perform a correction method for correcting outliers on an oligonucleotide microarray using PCA(principal component analysis), wherein the program performs the steps of:
(a) detecting an outlier in the oligonucleotide microarray data;
(b) constructing a first correlation structure model for model data to be used to correct the outlier by use of PCA;
(c) correcting a faulty probe data by projecting said first correlation structure model to the outlier.

18. The computer-readable medium of claim 17, wherein said step (a) comprises the steps of:
constructing a second correlation structure model of probes of each probe set by use of the PCA; and
detecting the outlier through calculating SPE index between the model value from said second correlation structure model and the raw data value.

19. The computer-readable medium of claim 17, wherein said first correlation structure model is the correlation structure of the probes included in probe set of each gene.

20. The computer-readable medium of claim 17, wherein said first correlation structure model and said second correlation structure model are covariance matrix comprising the correlation data of the probes included in each probe set.

21. A correction apparatus of an oligonucleotide microarray, said apparatus comprising:
a correlation structure model generator for constructing a correlation structure model indicating a correlation structure between probes of the oligonucleotide microarray data by use of the PCA; and
a data corrector for correcting a faulty probe data by projecting said correlation structure model to the outlier.

22. The correction apparatus of claim 21, wherein said correlation structure model generator generates a data matrix of each probe set of each gene for the oligonucleotide microarray data and constructs the correlation structure model of the probes included in the data matrix.

23. The correction apparatus of claim 22, wherein said correlation structure model is a covariance matrix comprising the correlation data of the probes.

24. The correction apparatus of claim 21, wherein said data corrector corrects data by performing a product of the data matrix including the outlier and said covariance matrix including the correlation data and iterates the data correction until the outlier data value converges.

25. A correction apparatus of an oligonucleotide microarray, said apparatus comprising:
an outlier extractor for detecting outlier from the oligonucleotide microarray data
a first correlation structure model generator for constructing a first correlation structure model for model data to be used to correct the outlier by use of the PCA; and
a data corrector for correcting a faulty probe data by projecting said first correlation structure model to the outlier.

26. The apparatus of claim 25, wherein said outlier extractor comprises:
means for constructing a second correlation structure model of probes of each probe set by use of PCA; and
means for detecting the outlier through calculating SPE index between the model value from said second correlation structure model and the raw data value.

27. The apparatus of claim 25, wherein said first correlation structure model is the correlation structure of the probes included in each probe set of genes.

28. The apparatus of claim 25, wherein said first correlation structure model and said second correlation structure model are covariance matrix comprising the correlation data of the probes included in each probe set.

29. The apparatus of claim 25, wherein the model data are selected from the samples of a group close to the value of the outlier.

30. The apparatus of claim 25, wherein said data corrector corrects data by performing a product of the data matrix including the outlier and said covariance matrix including the correlation data and iterates the data correction till the outlier data value converges.

31. A program being executed in a digital processing device to perform a correction method on an oligonucleotide microarray, wherein said program controls operations of the digital processing device to perform the correction method, said program performs the steps of:
(a) constructing a correlation structure model indicating a correlation structure between probes of the oligonucleotide microarray data by use of the PCA; and
(b) correcting a faulty probe data by projecting said correlation structure model to the outlier.

32. A program being executed in a digital processing device to perform a correction method on an oligonucleotide microarray, wherein said program controls operations of the digital processing device to perform the correction method, said program performs the steps of:
(a) detecting an outlier in the oligonucleotide microarray data;
(b) constructing a first correlation structure model for model data to be used to correct the outlier by use of PCA;
(c) correcting a faulty probe data by projecting said first correlation structure model to the outlier.
